(19) United States
(12) Patent Application Publication
(10) Pub. No.: US 2023/0047622 A1
(43) Pub. Date: Feb. 16, 2023

(54) VR-Based Treatment System and Method

(71) Applicant: Neurotechnology Pty. Ltd., Edgecliff, New South Wales (AU)

(72) Inventors: Wilfred Maurice Lax, Maroubra (AU); Dhani Christomo Sutanto, Leichhardt (AU)

(21) Appl. No.: 17/796,928

(22) PCT Filed: Feb. 3, 2021
(86) PCT No.: PCT/AU2021/050082

§ 371 Date: Aug. 2, 2022

(30) Foreign Application Priority Data
Feb. 3, 2020 (AU) 2020900282

(51) Int. Cl. A61M 21/02; G06F 3/01; G06T 19/00; G16H 30/40

(52) U.S. Cl. CPC A61M 21/02; G06F 3/011; G06T 19/006; G16H 30/40; A61M 2021/005

(57) ABSTRACT

An XR-based system (virtual reality, augmented reality, or mixed reality system), is provided to visualize and resolve at least one condition of a subject. A dynamic virtual representation of the subject's body is generated based on the captured physical traits and movement of the subject's body is captured by at least one motion tracking device, and rendered in the extended reality environment. The dynamic virtual representation is synchronized with the movement of the body of the subject, generating a virtual representation of at least one condition of the subject in response to one or more inputs, overlaying or rendering the virtual representation of the condition of the subject on the virtual representation of the body of the subject, and receiving and processing one or more inputs representing one or more attributes of the condition to adjust the virtual representation of the condition of the subject in the extended reality environment.

This application relates to and claims priority from Australian Provisional Application No. 2020900282, entitled “VR-based treatment system and method”, filed on 3 Feb. 2020, the contents of which are hereby incorporated by reference in their entirety.

### FIELD OF THE INVENTION

The invention relates to a VR-based treatment system and method, and in particular to a VR-based treatment system and method for the treatment of a health condition, including the treatment or management of pain. More generally, the invention relates to an XR-based treatment system and method.

### BACKGROUND OF THE INVENTION

Recent research indicates that virtual reality or VR can effectively be used in the field of pain management. The analgesic properties of VR have been mainly attributed to its distractive capacity. It has also been recognised that immersive VR is effective in diminishing sensations of pain. VR-based interventions have been used to decrease acute pain amongst individuals undergoing painful medical procedures, including treatment of burns injuries, dental pain and physical therapy for blunt force trauma and burns injuries.

The effective use of VR or more broadly XR in the treatment of chronic or persistent pain is less well documented, and there is accordingly a need for VR/XR-based systems for the effective treatment of such pain, as well as for the treatment of mental and physical health problems in general in an immersive VR/XR-based environment.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect of the disclosure there is provided a virtual reality-based treatment system for performing treatment on at least one condition of a subject, including;

a virtual reality device arranged to be fitted to the subject and for immersing the subject in a virtual reality environment;

at least one tracking camera configured to capture physical traits and movement of the body of the subject;

a processor communicating with the virtual reality device and the at least one tracking camera;

a monitor in communication with the processor, and including a user interface, wherein

the processor is programmed to:
generate a dynamic virtual representation of the body of the subject based on the captured physical traits and movement of the body of the subject, and to render the virtual representation of the body in the virtual reality environment via the virtual reality device, wherein the dynamic virtual representation is synchronised with the movement of the body of the subject;
generate a virtual representation of the at least one condition of the subject in response to one or more inputs;
overlay or render the virtual representation of the condition of the subject on the virtual representation of the body of the subject; and
receive and process one or more inputs representing one or more attributes of the condition to adjust the virtual representation of the condition of the subject in the virtual reality environment to thereby assist the subject to visualise and resolve the condition.

In another aspect there is provided a method of performing a treatment on at least one condition of a subject in an immersive virtual reality environment comprising:
capturing physical traits and movement of the body of the subject;
generating a dynamic virtual representation of the body of the subject based on the captured physical traits and movement of the body of the subject;
rendering the dynamic virtual representation of the body of the subject in the virtual reality environment, wherein the dynamic virtual representation is synchronised with the movement of the body of the subject;
generating a dynamic virtual representation of the at least one condition of the subject in response to one or more inputs;
overlaying or rendering the virtual representation of the condition of the subject on the virtual representation of the body of the subject; and
receiving and processing one or more inputs representing one or more attributes of the condition to adjust the virtual representation of the condition of the subject in the virtual reality environment to thereby assist the subject to visualise and resolve the condition.

The method may include generating virtual representations of multiple layers or components of the virtual body selected from at least two of a skin layer or component, a muscle layer or component, a nerves layer or component, an organs layer or component, a vascular layer or component, a respiratory layer or component and a skeleton layer or component, and enabling switching between virtual representations of the layers or components.

The visual representations of the attributes of the condition may include at least two of location, start point, end point, depth, intensity, size, speed, direction, frequency, temperature as indicated by colour and type as indicated by symbols.

The captured physical traits may include at least three of body shape, face shape, skin colour, hair colour/style, eye colour, height, weight, and gender.

The step of generating virtual representations of the body of the subject may include generating selectable or interchangeable direct self and mirror self-representations of the subject, the mirror representations of the subject being generated by generating an inverse image of the subject as opposed to using a virtual mirror plane.

The method may include generating a virtual representation of the body of a host or treatment provider, typically based on the captured physical traits and movement of the body of the host, and rendering the virtual representation of the body of the host in the virtual reality environment;

The condition may include pain, chronic pain, a physical or mental ailment or disability, including various levels of paralysis or palsy, and may further include a physical or mental state which requires enhancing or therapy, such as muscle condition, mental acuity, or stress. The disability may relate to amputees, and the treatment may include mental and physical training of amputees, including emulating their lost limb to train their nerves and muscles before using artificial limbs.

The disclosure extends to a system wherein the processor is programmed to implement any of the above methods.

The disclosure extends further to a non-transient storage medium readable by a processor, the storage medium storing a sequence of instructions or software executable by the processor to cause the processor to perform the any of the above methods.

The disclosure extends to a non-transient storage medium in which the sequence of instructions or software includes:

a virtual subject creator module to capture physical traits of the body of a subject and render a virtual subject including those traits;

a virtual subject controller module to capture movement of the body of the subject and render a moving virtual subject using the virtual subject from the virtual human creator module;

a virtual condition module to generate a virtual representation of at least one condition of the subject in response to one or more inputs, and layer it on the moving virtual subject; and

a virtual environment module for providing a selectable virtual environment for the subject.

The software may include a virtual camera module for generating a selection of views or perspectives of the subject being treated.

The disclosure further extends to a virtual reality-based treatment system for performing treatment on at least one condition of a subject, including:

a virtual reality device arranged to be fitted to the subject and for immersing the subject in a virtual reality environment;

at least one tracking camera configured to capture physical traits and movement of the body of the subject;

a processor communicating with the virtual reality device and the at least one tracking camera;

a monitor in communication with the processor, and including a user interface, wherein

the processor is programmed with a plurality of software modules to generate a dynamic virtual representation of the body of the subject based on the captured physical traits and movement of the body of the subject, and to render the virtual representation of the body in the virtual reality environment via the virtual reality device, wherein the dynamic virtual representation is synchronised with the movement of the body of the subject, the software modules including;
a virtual subject creator module to capture physical traits of the body of a subject and render a virtual subject including those traits;
a virtual subject controller module to capture movement of the body of the subject and render a moving virtual subject using the virtual subject from the virtual human creator module;
a virtual condition module to generate a virtual representation of the at least one condition of the subject in response to one or more inputs, and layer it on the moving virtual subject; and
a virtual environment module for providing a selectable virtual environment for the subject.

The disclosure further extends to an extended reality (XR) based treatment system for performing treatment on at least one condition of a subject, including;

an XR device arranged to be fitted to the subject and for engaging the subject in an XR environment;

at least one motion tracking device configured to capture physical traits and movement of the body of the subject;

a processor communicating with the XR device and the at least one motion tracking device;

a monitor in communication with the processor, and including a user interface, wherein

the processor is programmed to:
generate a dynamic virtual representation of the body of the subject based on the captured physical traits and movement of the body of the subject, and to render the virtual representation of the body in the XR environment via the XR device, wherein the dynamic virtual representation is synchronised with the movement of the body of the subject;
generate a virtual representation of the at least one condition of the subject in response to one or more inputs;
overlay or render the virtual representation of the condition of the subject on the virtual representation of the body of the subject;
receive and process one or more inputs representing one or more attributes of the condition to adjust the virtual representation of the condition of the subject in the XR environment to thereby assist the subject to visualise and resolve the condition.

The extended reality (XR) based treatment system includes the processor being programmed to implement any of the above methods.

The disclosure further extends to an extended reality (XR) based treatment system for performing treatment on at least one condition of a subject, including;
an XR device arranged to be fitted to the subject and for engaging the subject in an XR environment;
at least one motion tracking device configured to capture physical traits and movement of the body of the subject;
a processor communicating with the XR device and the at least one motion tracking device;
a monitor in communication with the processor, and including a user interface, wherein

the processor is programmed with a plurality of software modules to generate a dynamic virtual representation of the body of the subject based on the captured physical traits and movement of the body of the subject, and to render the virtual representation of the body in the XR environment via the XR device, wherein the dynamic virtual representation is synchronised with the movement of the body of the subject, the software modules including;
a virtual subject creator module to capture physical traits of the body of a subject and render a virtual subject including those traits;
a virtual subject controller module to capture movement of the body of the subject and render a moving virtual subject using the virtual subject from the virtual human creator module;
a virtual condition module to generate a virtual representation of the at least one condition of the subject in response to one or more inputs, and layer it on the moving virtual subject; and
an XR environment module for providing a selectable XR environment for the subject.

The disclosure further extends to a method of performing a treatment on at least one condition of a subject in an extended reality (XR) environment comprising:
capturing physical traits and movement of the body of the subject;
generating a dynamic virtual representation of the body of the subject based on the captured physical traits and movement of the body of the subject;
rendering the dynamic virtual representation of the body of the subject in the XR environment, wherein the dynamic virtual representation is synchronised with the movement of the body of the subject;
generating a virtual representation of the at least one condition of the subject in response to one or more inputs;
overlaying or rendering the virtual representation of the condition of the subject on the virtual representation of the body of the subject; and
receiving and processing one or more inputs representing one or more attributes of the condition to adjust the virtual representation of the condition of the subject in the XR environment to thereby assist the subject to visualise and resolve the condition.

The extended reality (XR) based treatment system or method may be selected from the group comprising at least one of virtual reality (VR), augmented reality (AR) and mixed reality (MR).

The XR-based treatment system may further includes:

a database for collecting historical data; and

a machine learning processor;
wherein the historical data is used to train the machine learning processor so that the machine learning processor generates one or more executable treatment actions based on the one or more inputs representing one or more attributes of the at least one condition of the subject; and
wherein the generated one or more executable treatment actions are provided to the processor for visualisation and resolving the condition.

The historical data may include one or more of XR hardware data, XR software data, user data and host data. The generated one or more executable treatment actions may be fed back to the database.

The trained machined learning processor further generates analytical data to evaluate one or more treatment results, and wherein the generated analytical data is fed back to the database.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1a shows a schematic block diagram of one embodiment of a VR-based treatment system;

FIG. 1b is a schematic block diagram of a computer processing system forming part of the VR-based system of FIG. 1a and configurable to perform various features of a VR-based treatment method of the present disclosure;

FIG. 1c is a schematic block diagram of a computer network including the computer processing system of FIG. 1b;

FIG. 2 shows a workflow diagram incorporating an embodiment of a VR-based treatment method;

FIG. 3 shows one embodiment of a host user interface;

FIG. 4a shows a pop-up menu forming part of the interface of FIG. 3 for changing camera and headset settings;

FIG. 4b shows a pop-up menu forming part of the interface of FIG. 3 for allowing adjustment of the user's view

FIG. 4c shows a controller and part of the interface of FIG. 3 for selecting pain type;

FIG. 4d shows a controller and part of the interface of FIG. 3 for selecting pain attributes including magnitude and speed;

FIGS. 4e, 4f, 4g, 4h and 4k show representations of respective skin, muscle, nerve, organ and skeleton layers selectable by the host user interface;

FIG. 4m shows a pain point selector part of the interface of FIG. 3 for selecting pain points;

FIG. 4ma shows an experience mode from a user perspective in which a self view of a virtual image of a user's arm is shown as well as a reflected view of the user;

FIG. 4n shows a virtual representation of a user showing the nervous system layer and a wrist-focused pain point with associated pain particles;

FIG. 5 shows a schematic block diagram of an embodiment of the hardware and software components of the VR-based system; and

FIG. 6 shows a schematic block diagram of an embodiment of an XR-based system implemented with a machine learning software module.

While the invention as claimed is amenable to various modifications and alternative forms, specific embodiments are shown by way of example in the drawings and are described in detail. It should be understood, however, that the drawings and detailed description are not intended to limit the invention to the particular form disclosed. The intention is to cover all modifications, equivalents, and alternatives falling within the spirit and scope of the present invention as defined by the appended claims. For example, it will be appreciated that the VR technology described in this disclosure is one example of extended reality (XR) technologies, wherein the letter “X” represents a variable for any current or future computer altered reality technologies. In other words, it will be appreciated that the disclosed treatment system and method may be implemented with other real-and-virtual combined environments and human-machine interactions generated by computer technology and wearables (i.e. XR technologies), such as augmented reality (AR), mixed reality (MR) or any combination of VR, AR and MR.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

In the following description numerous specific details are set forth in order to provide a thorough understanding of the claimed invention. It will be apparent, however, that the claimed invention may be practiced without these specific details. In some instances, well-known structures and devices are shown in block diagram form in order to avoid unnecessary obscuring.

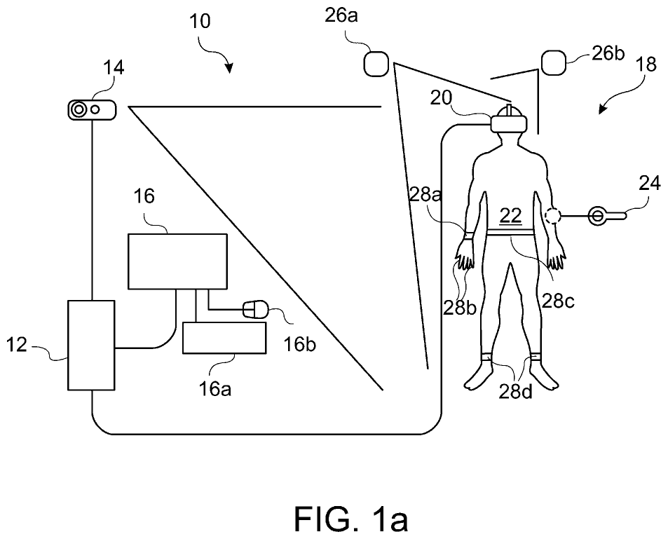

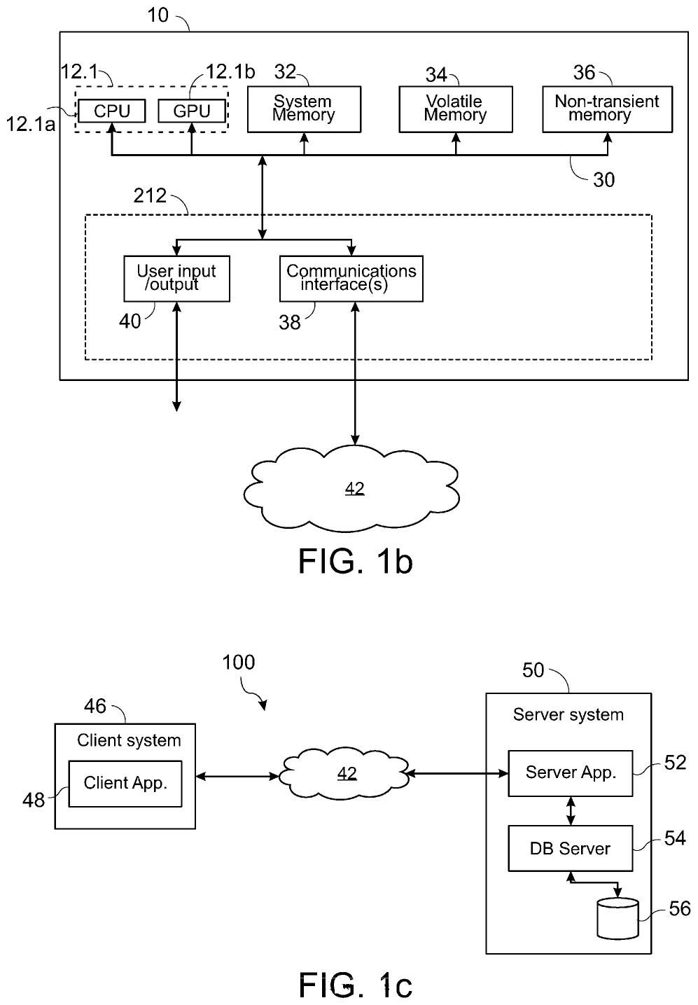

Referring first to FIG. 1a, one embodiment of a VR-based system 10 includes at its heart a computer processing system 12 in communication with at least one tracking camera 14. The tracking camera may for example include a Microsoft Kinect 2.0 camera, a Microsoft Azure Kinect camera, or other commercially available tracking cameras to track the user's body and movement. The computer processing system 12 also communicates with a host monitor 16 with input devices/means in the form of the keyboard 16a and a mouse 16b. Other input means may include a touchscreen enabled monitor, a touchpad, and any form of remote controlled device including a gaming console.

The system further includes a VR arrangement 18 including a VR headset 20 worn by a user 22, an associated VR controller 24 which also acts as an input device, and VR trackers 26a and 26b. The VR headset may be selected from a number of commercially available headsets, including for example an HTC® Vive Pro headset or a Microsoft® Mixed Reality headset with corresponding trackers, in the present example HTC Vive Pro® trackers, and a corresponding HTC Vive Pro or Microsoft Mixed Reality controller 24. The trackers may include stationary trackers, such as those indicated 26a and 26b, which are configured to track the movement of the headset 20, as well as individual body trackers used to track the movement of parts of the body, such as wrist, finger, waist, or ankle trackers 28a, 28b, 28c and 28d respectively, which include corresponding straps or belts.

FIG. 1b shows a block diagram of the computer processing system 12 configurable to implement embodiments and/or features described herein. It will be appreciated that FIG. 1b does not illustrate all functional or physical components of a computer processing system. For example, no power supply or power supply interface has been depicted, however system 12 will either carry a power supply or be configured for connection to a power supply (or both). It will also be appreciated that the particular type of computer processing system will determine the appropriate hardware and architecture, and alternative computer processing systems suitable for implementing features of the present disclosure may have additional, alternative, or fewer components than those depicted.

Computer processing system 12 includes at least one processing unit 12.1 which may in turn include a CPU 12.1a and a GPU 12.1b. The CPU 12.1a may include at least an Intel core i7 8700 processor, preferably a 9700 processor or the like, with the GPU 12.1b including at least a GTX 1080ti processor, preferably a RTX 2080ti or a Titan RTX processor. It will be appreciated that the abovementioned hardware, including the VR hardware, may be superseded or updated on a regular basis with hardware and technologies having improved specifications, and it is within the scope of this disclosure to include such improved and updated hardware.

The processing unit 12.1 may be a single computer processing device (e.g. a combined central processing unit and graphics processing unit, or other computational device), or may include a plurality of computer processing devices, such as a separate CPU and GPU as described above. In some instances all processing will be performed by processing unit 12.1, however in other instances processing may also be performed by remote processing devices accessible and useable (either in a shared or dedicated manner) by the system 12.

Through a communications bus 30 the processing unit 12.1 is in data communication with a one or more machine readable storage (memory) devices which store instructions and/or data for controlling operation of the processing system 12. In this example system 12 includes a system memory 32 (e.g. a BIOS), volatile memory 34 (e.g. random access memory such as one or more RAM or DRAM modules with a minimum of 32 MB RAM), and non-volatile memory 36 (e.g. one or more hard disk or solid state drives)

System 12 also includes one or more interfaces, indicated generally by 38, via which system 12 interfaces with various devices and/or networks. Generally speaking, other devices may be integral with system 12, or may be separate. Where a device is separate from system 12, connection between the device and system 12 may be via wired or wireless hardware and communication protocols, and may be a direct or an indirect (e.g. networked) connection.

Wired connection with other devices/networks may be by any appropriate standard or proprietary hardware and connectivity protocols. For example, system 12 may be configured for wired connection with other devices/communications networks by one or more of: USB; FireWire; eSATA; Thunderbolt; Ethernet; OS/2; Parallel; Serial; HDMI; DVI; VGA; SCSI; AudioPort. Other wired connections are possible.

Wireless connection with other devices/networks may similarly be by any appropriate standard or proprietary hardware and communications protocols. For example, system 12 may be configured for wireless connection with other devices/communications networks using one or more of: infrared; BlueTooth; WiFi; near field communications (NFC); Global System for Mobile Communications (GSM), Enhanced Data GSM Environment (EDGE), long term evolution (LTE), wideband code division multiple access (W-CDMA), code division multiple access (CDMA). Other wireless connections are possible.

Generally speaking, and depending on the particular system in question, devices to which system 12 connects—whether by wired or wireless means—include one or more input devices to allow data to be input into/received by system 12 for processing by the processing unit 12.1, and one or more output devices to allow data to be output by system 12. Example devices are described below, however it will be appreciated that not all computer processing systems will include all mentioned devices, and that additional and alternative devices to those mentioned may well be used.

For example, system 12 may include or connect to one or more input devices by which information/data is input into (received by) system 12. Such input devices may include keyboards, mice, trackpads, microphones, accelerometers, proximity sensors, GPS devices and the like. System 12 may also include or connect to one or more output devices controlled by system 12 to output information. Such output devices may include devices such as a CRT displays, LCD displays, LED displays, plasma displays, touch screen displays, speakers, vibration modules, LEDs/other lights, and such like. System 12 may also include or connect to devices which may act as both input and output devices, for example memory devices (hard drives, solid state drives, disk drives, compact flash cards, SD cards and the like) which system 12 can read data from and/or write data to, and touch screen displays which can both display (output) data and receive touch signals (input). FIG. 1 shows just one exemplary implementation.

System 12 may also connect to one or more communications networks (e.g. the Internet, a local area network, a wide area network, a personal hotspot etc.) to communicate data to and receive data from networked devices, which may themselves be other computer processing systems.

System 12 may be any suitable computer processing system such as, by way of non-limiting example, a server computer system, a desktop computer, a laptop computer, a netbook computer, a tablet computing device, a mobile/smart phone, a personal digital assistant, a personal media player, a set-top box, and a games console.

Typically, system 12 will include at least user input and output devices 40, which may be of the type described with reference to FIG. 1 and a communications interface 42 for communication with a network such as network 42.

System 12 stores or has access to computer applications (also referred to as software or programs)—i.e. computer readable instructions and data which, when executed by the processing unit 12.1, configure system 12 to receive, process, and output data. Instructions and data can be stored on non-transient machine readable medium accessible to system 12. For example, instructions and data may be stored on non-transient memory 36. Instructions and data may be transmitted to/received by system 12 via a data signal in a transmission channel enabled (for example) by a wired or wireless network connection.

Applications accessible to system 12 will typically include an operating system application such as Microsoft Windows®, Apple OSX, Apple IOS, Android, Unix, or Linux.

System 12 also stores or has access to applications which, when executed by the processing unit 12.1, configure system 12 to perform various computer-implemented processing operations described herein. For example, and referring to the networked environment of FIG. 1c above, client system 46 includes a client application 48 which configures the client system 46 to perform the described client system operations, and server system 50 includes a server application 52 which configures the server system 50 to perform the described server system operations. The server application 52 communicates with a database server 54 which enables the storage and retrieval of data stored in a database 56, which may be a distributed or cloud-based database.

In some cases part or all of a given computer-implemented method will be performed by system 12 itself, while in other cases processing may be performed by other devices in data communication with system 12.

The client application 48 is designed, in combination with the hardware described in FIG. 1a, to immerse the user in a virtual environment where they see a virtual representation of themselves. This representation is designed to be as accurate as feasible with regard to height and body type with the aid of the body tracking camera 14. It has been established that sufficient user identity with the virtual representation can be achieved without providing strict anatomical accuracy or identical facial features. One aspect which contributes significantly to this is the accurate tracking of actual body movements of the user by their virtual representation with minimal latency (i.e. a delay of typically less than 90 ms, more typically 80-88 ms). This provides the user with a subjective impression of simultaneity or synchronicity which enhances the immersive experience and the identity of the user with their real and mirror selves. The virtual representation is applied to the main elements of the real self and the mirror self.

The application 48 helps the user to visualise their condition and also assists the host, who is typically a trained psychologist, therapist or clinician, to help educate the user and to start the condition management therapy session.

The application is also designed to display various visual representations of the user, including a high level or impressionist representation of the gender of the user, which is confined to male and female, as well as various layers of the user's body, including a skin layer, muscle layer, nerves layer, and internal organs layer, and a skeletal layer. Additional layers may include a vascular or cardio-vascular layer, and a respiratory layer.

The application is further designed to provide a symbolic visual representation of the condition, such as pain, which is preferably a dynamic representation, and is overlaid on the virtual visual representation of the user. This is typically achieved by the host using the virtual reality controller 24. The application may further provide a virtual visualisation of the host and the controller which is viewable through the virtual reality headset 20 as well as the monitor 16. The user and host are immersed in a virtual reality environment which may initially include an on-boarding environment followed by other immersive environments described hereinafter.

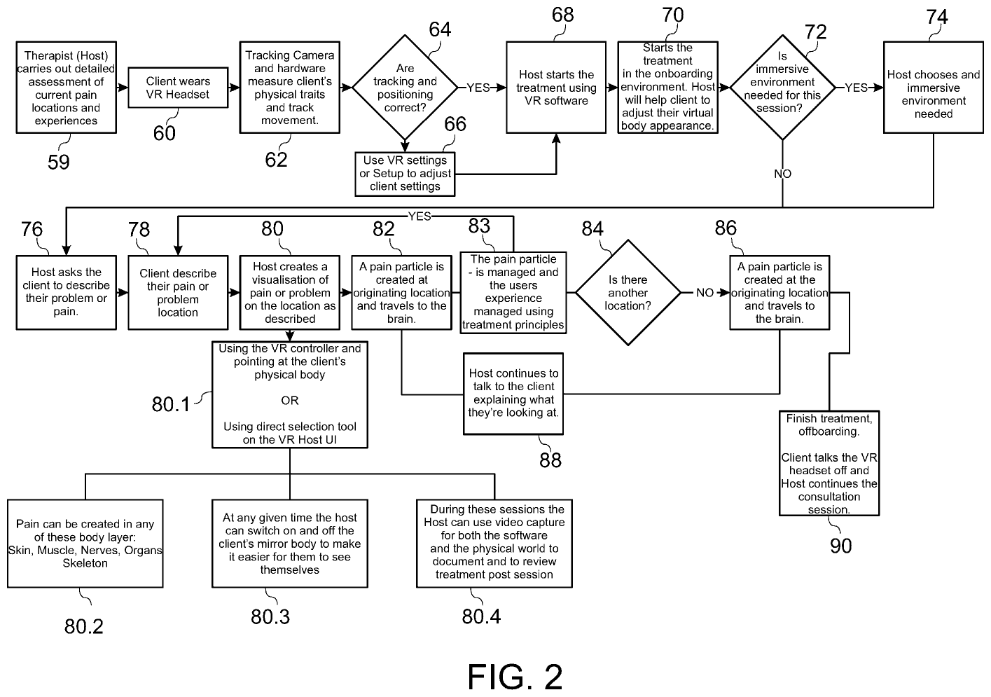

Referring now to FIG. 2, a flow diagram is shown incorporating exemplary steps used in an embodiment of a chronic pain treatment method of the disclosure. At initial step 59, the host carries out a detailed assessment of the current pain locations and experiences of the user or client/patient, and documents these. At step 60, the client or user 22 then dons the VR headset 20. Then at step 62, the tracking camera 14 and associated hardware measure the user's physical traits and track movement of the user. A check is conducted at 64 to see if the tracking and positioning of the headset is correct. If not, the host adjusts the VR settings via the host monitor 16, as is shown at 66.

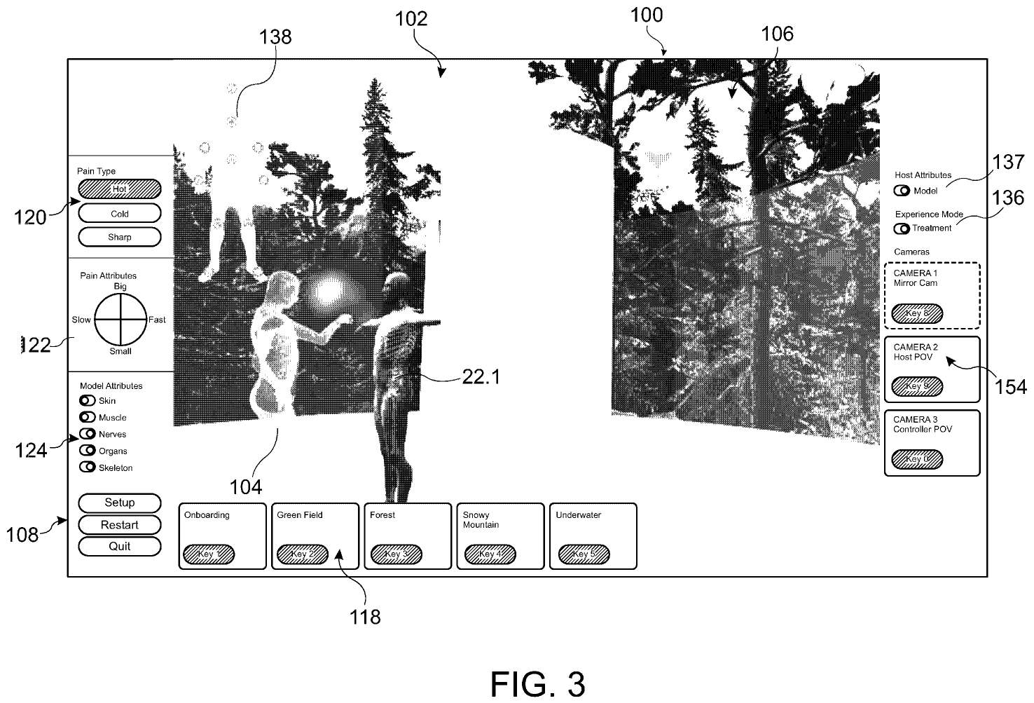

FIG. 3 shows one embodiment of a host user interface or GUI 100 which is generated by the application on the host monitor 16. The host interacts with the host user interface via input devices including controller 24, keypad 16a and mouse 16b. The host user interface 100 includes a central display 102 which provides the host's perspective of the virtual reality environment in which the user 22 is immersed, including a virtual representation 22.1 of the user as well as an optional virtual representation 104 of the host, which may also be viewed by the user through the VR headset 20. The virtual body representation of the host may be similar to that of the user, but may also include natural elements such as fire or water, or in the case of treating children the host may adopt the appearance of an avatar in the form of a friendly robot or a familiar fantasy character.

In the present example, the user 22 and host 104 are immersed in a forest environment 106. The central display 102 is surrounded by a series of selection inputs which are controlled by the host in consultation with the user to customise the treatment of the user and to optimise the experience of the user during a treatment session.

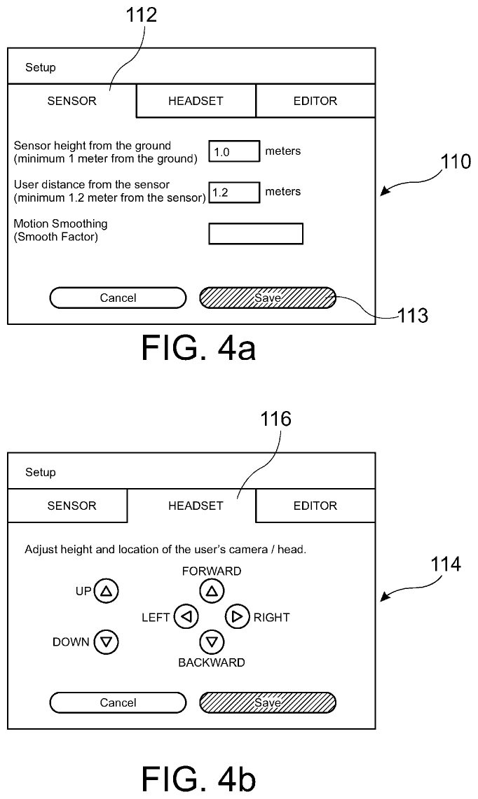

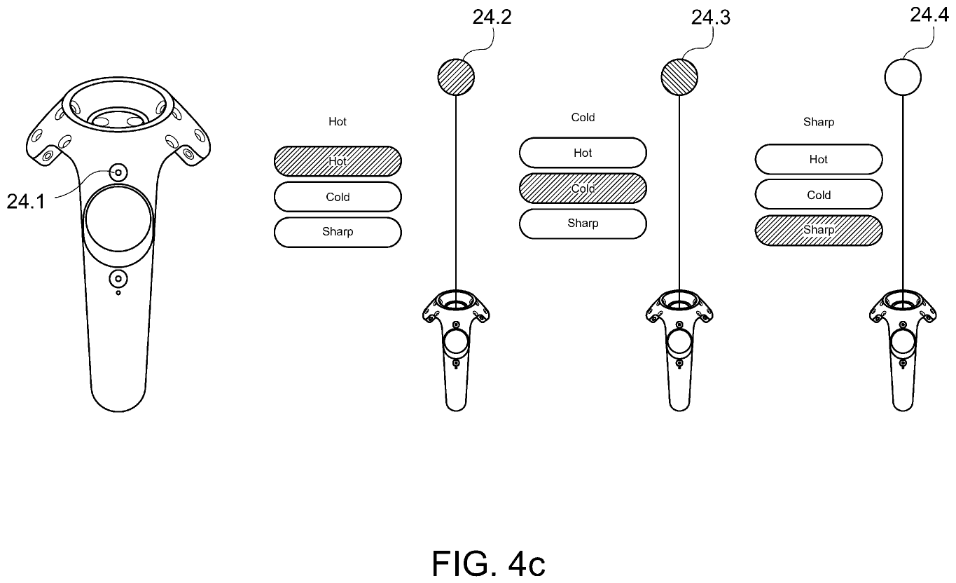

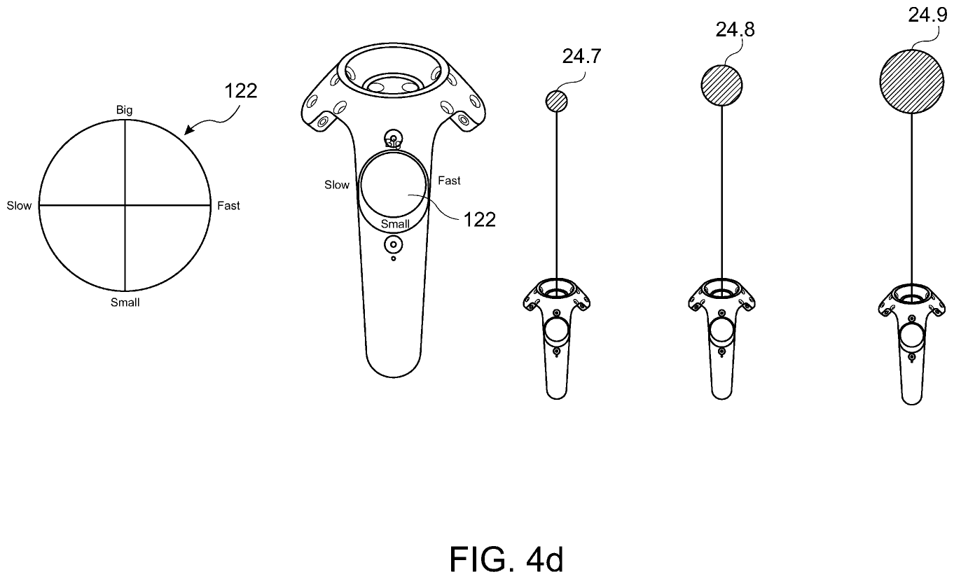

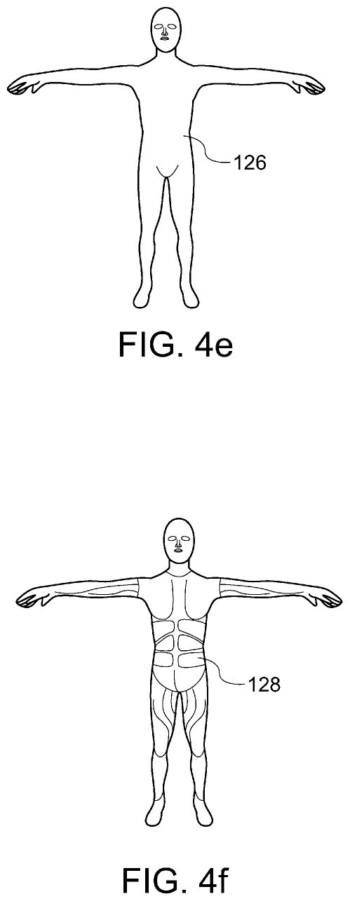

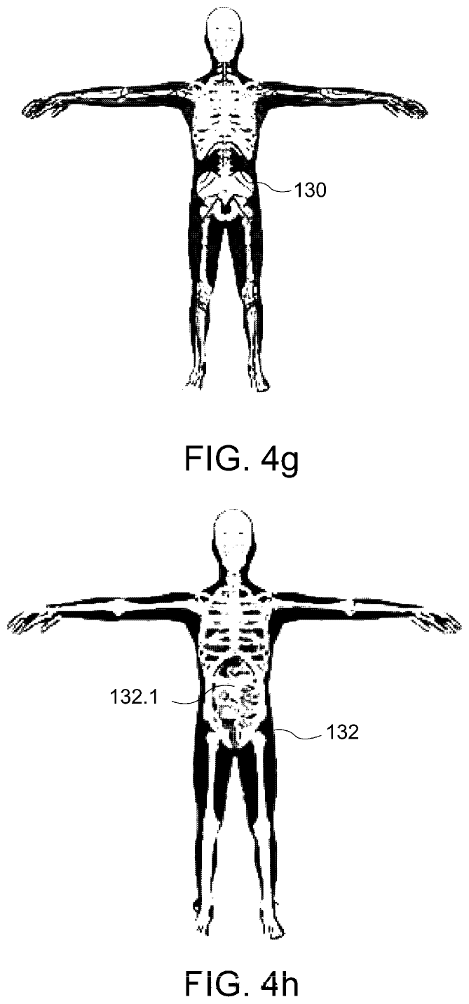

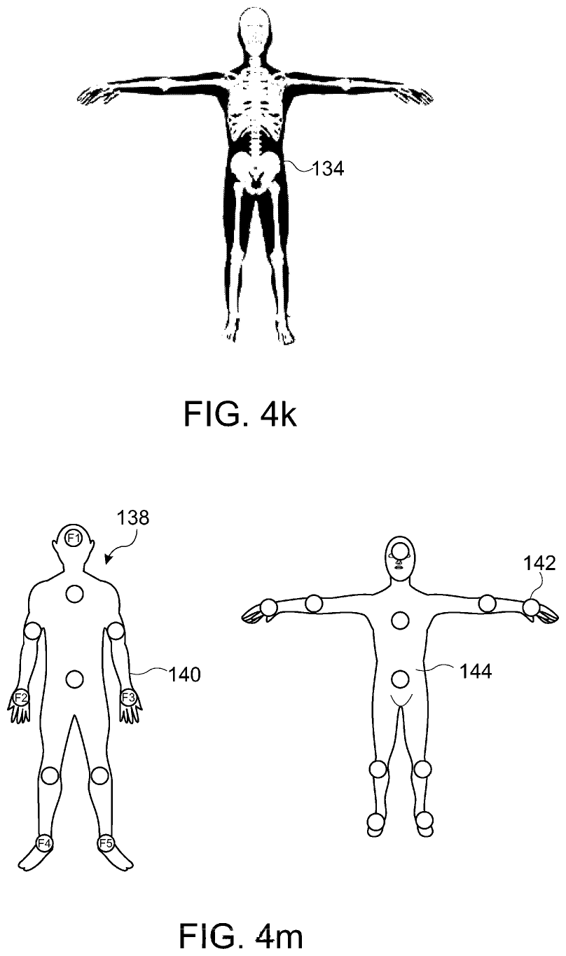

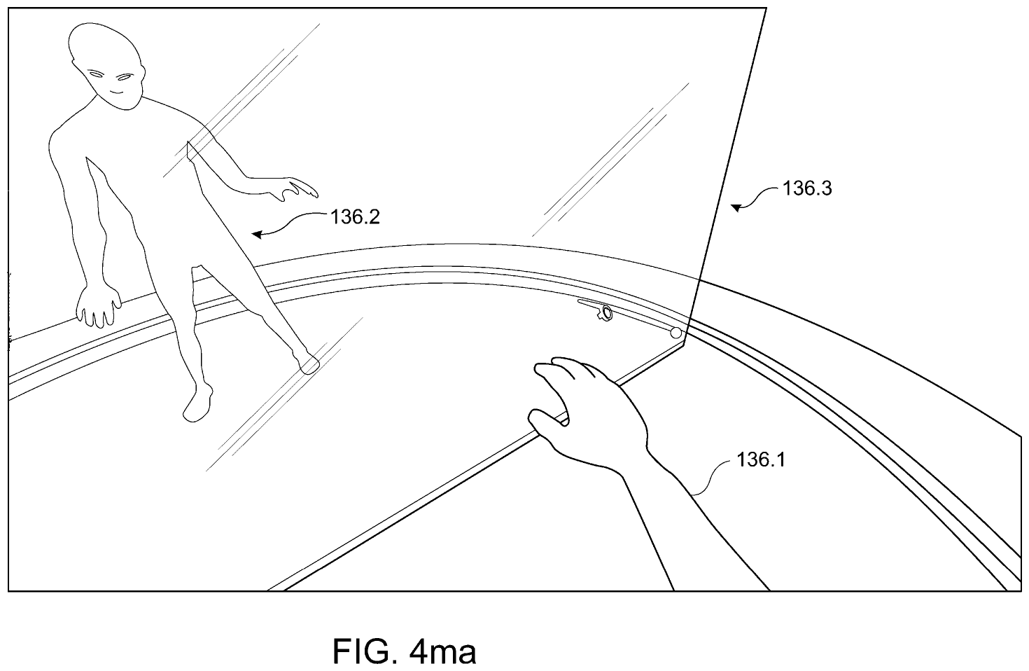

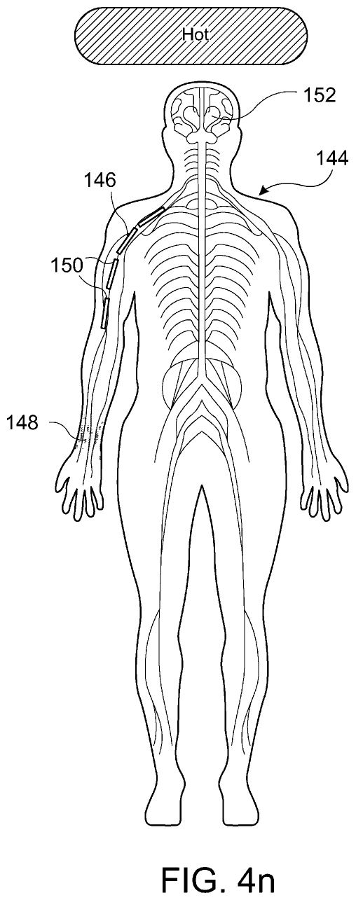

Software settings inputs 108 provide respective setup, restart and quit options operable via one of the input devices. Activation of the setup or restart settings opens a pop-up menu 110 shown in FIG. 4a via sensor tab 112 which allows the host to commence step 66 of FIG. 2 by adjusting the height of the motion tracking camera 14 from the ground to a height greater than one meter and the user distance from the motion sensor camera 14, a minimum of 1.2 meters, as well as to enter a motion smoothing factor which determines how frequently the camera 14 updates the user's position. The save button 113 is used to save the settings.

An additional headset pop-up menu 114 is then activated via headset tab 116, as is shown in FIG. 4b. This setting allows the host to adjust the virtual position of the headset so that the user's VR view is correct relative to their body. This is achieved by using the indicated up, down, left, right, and forward and backward buttons, to adjust the position of the user's virtual camera so that the self and mirror virtual images that are generated of the user correspond as closely as possible with the user's actual position, with the final adjusted position being saved. The host then commences treatment at 68 using the VR software, starting treatment in the on-boarding environment at step 70. The on-boarding environment is selectable via Key 1 of an environment selector 118, which includes additional Keys 2, 3, 4 and 5 for respectively enabling the selection of green field, forest, snowy mountain and underwater environments. It will be appreciated that many other possible environments may be generated. The keys may be activated via any of the aforementioned input devices.

At step 72, a decision is made as to whether an immersive environment is required for the session. If so, the host chooses one of the above-mentioned immersive environments at step 74, potentially taking user preferences into account, which in this case is the forest environment 106. If not, the host commences directly to 76. The weather conditions associated with the environments may also be relevant to treatment. For example a cold (snowy mountain) environment may be effective in the treatment of burns or burning pain.

The host then at 76 asks the user to describe their condition/problem, which in this example is pain-related. This may supplement or replace the initial assessment at step 59. At step 78, the user/client then describes the nature of their pain/problem and its location. The exchange between the user and the host may conveniently be verbal, but may also be in writing, and may in addition operate in an environment where the user and the host are not in the same location, and the written or verbal communication is over a suitable communications network.

At step 80, the host then creates a visualisation of the pain or problem at the described location. This may be achieved at step 80.1 using the VR controller 24 which the host points at the relevant location on the user's body, or by using a direct selection tool on the host interface 100 including the monitor 16 and inputs 16.1 and 16.2.

A pain type selector including menu 120 is displayed on the monitor, including indicated hot, cold and sharp types of pain. It will be appreciated that other pain types may also be indicated for selection, such as dull, or throbbing. Referring to FIG. 4c, the controller 24 has a menu button 24.1 which is repurposed as a pain type button used to select one of the above pain types. The pain types may in turn be represented by colours, with hot, cold and sharp pain types being represented by red, blue and purple colours respectively. These colours may be indicated by red, blue and purple orbs 24.2, 24.3 and 24.4 extending from the tip of the controller 24 in the VR environment. Pain types may also be represented by objects or phenomena associated with creating that type of pain. For example flames/red hot pokers may be used to indicate burning pain, knives or needles or lightning bolts to indicate sharp and intense pain, hammers and clubs to indicate dull throbbing pain, and pincers to indicate localised surface pain.

The host user interface 100 also includes a pain attribute selector including a pain attribute menu or circular icon 122 with magnitude of pain from small to big as indicated by the user on the vertical axis and pain velocity or speed from slow to fast on the horizontal axis. Pain velocity may be used to indicate pain frequency in the case of a throbbing pain for instance or pain velocity in the case of a shooting pain. As is shown in FIG. 4d, the circular touchpad 24.6 on the controller is repurposed as a pain attribute selector, with the host altering the pain magnitude by scrolling and pressing on the touchpad 24.6, which operates in the same way as the circular icon 122. In FIG. 4d it can be seen how in the VR environment different sized orbs 24.7, 24.8 and 24.9 are used to indicate magnitude of pain.

The host interface 100 further includes a model attribute selector including a model attribute menu 124 for enabling the selection of layers of the user's VR body to be selected at step 80.2. These include a skin layer 126 of FIG. 4e which is the default or starting state of the user's VR body. The skin layer is gender specific, without the associated anatomical detail, and the overall body type and height represents the body type and height of the user, based on the images of the user captured by the tracking camera 14 and processed by the computer system 12. The ability of the user to identify with their virtual selves is enhanced by accurate representations of body height and type.

The virtual representation of FIG. 4f shows a second muscle layer 128 with representations of the muscles on the client's body, which may be adjusted based on body type so that they conform with the skin layer.

FIG. 4g shows a third nerves layer 130 which is used to show how pain travels through the body in response to the user's description of that pain in the manner previously described. The nerves layer 130 is scaled to conform with the size and shape of the user's body.

FIG. 4h shows a fourth organs layer on 132. If the pain originates from a particular organ, this organ is highlighted. For example, in FIG. 4a, the digestive system 132.1 is highlighted, and the pain is represented as travelling from the digestive system to the brain. It will be appreciated that various other organs can be displayed in the same manner and highlighted when relevant to the pain experienced by the user.

FIG. 4k shows a fifth skeleton layer 134, which is the deepest layer. Bone or joint pain can be illustrated and localised using this layer. The various layers enhance the user experience by allowing the user to locate their pain more precisely in 3D as well as providing a realistic virtual representation of the affected body part and its relationship with the pain being experienced by the user.

As is shown at step 80.3, the host can turn the user's mirror body on and off to make it easier for the user to see themselves by looking down when wearing the VR headset 20 to see a virtual representation of their arms and front portion of their bodies co-located with their real bodies, when both moving and still. This is achieved by operating an experience mode toggle 136 in FIG. 3 in which the host is able to toggle between the default self view and the experience mode or mirror view in which the user is able to see both views.

In FIG. 4ma, the experience mode is shown in which a self view of a virtual image of a user's arm 136.1 is shown as well as a reflected view of the entire body of the user 136.2 in a virtual mirror 136.3 as experienced by the user when wearing the VR headset. The combination of the self and mirror views serves to reinforce the user's immersion in that the user is able to view themselves both directly and when reflected. Because the self and reflected views are dynamically synchronised with the actual movement of the user this gives the user an even more immersive experience when moving by enhancing the user perception that the self and mirror views are embodiments of the user.

As previously described, in addition to varying the user view of their body, the host may also include a virtual image of themselves or a fantasy representation thereof. This is achieved by operating a host attribute toggle 137.

As indicated at 80.4, the host can use video to capture both real and virtual images of the user and host where applicable to review treatment protocols after the treatment session. This may be securely stored in the database 56.

At step 82, a pain particle or particles are created at the originating location of the pain and shown travelling to the brain. This is achieved using a direct point selector shown at 138 in FIG. 3 and FIG. 4m. The host may use shortcut keys, such as F1, F2, F3, F4 and F5 on the keypad to access pain points on the body outline 140 of the direct point selector, corresponding to points 142 on the virtual body of the user 144. The controller 24 may be used to more accurately pinpoint the exact location of pain points on the user's body, which may include the initial step of selecting an appropriate body layer. The pain type and attribute are also selected in the manner previously described, and this influences the size, colour and frequency of the pain point or zone as well as of the pain particles travelling to and/or from the pain point to the user's brain.

At step 83 the pain particles are configured and the experience of the user is managed by the host using treatment principles including cognitive behaviour therapy, learning therapy, neuroplasticity, and pain therapy in the VR environment that has been established.

At step 84 the user is asked if there are any other pain locations. If the answer is positive, the process reverts to step 78 at which the user describes the location and nature of the pain which is then converted by the host into a form which can be readily visualised. At step 86 pain particles continue to be created at the originating location(s) and are shown travelling to the brain. At step 88, the host continues to explain to the user what they're looking at and where necessary, adjustments may be made to the visualisations depending in some instances on user feedback.

FIG. 4n shows a static presentation of a virtual representation of a user 144.1 showing the nervous system 146 and a wrist-focused pain point 148 which is coloured red to represent burning pain, with pain particles 150 travelling to and from the brain 152. It will be appreciated that the pain particles are dynamically represented with variations in speed and/or frequency used to indicated the nature of the pain. In some embodiments the representation of the pain point or zone as well as the representation of the pain particles is dynamically varied to indicate, for example, an easing of the pain. This may be achieved by decreasing the magnitude of the zone and/or the pain particles, by changing the colour of the zone and pain particles from red to blue for example, and/or by slowing down the speed or frequency pain particles. In some embodiments, the pain point was zone and pain particles may be caused to fade away, again to create an illusion of reduced pain.

At step 90, treatment is completed (a session would typically take 15 to 20 minutes) and the user is off-boarded by removing the VR headset. The host then continues with the consultation session.

By virtue of three virtual cameras, CAMERAS 1, 2 and 3, the host is able to change their point of view of the user within the virtual world. This is achieved using camera selector interface 154 which in the present example uses Key 8 of the keypad to select the main mirror CAMERA1 providing a reflected or mirror perspective from the user's point of view, Key 9 to select the virtual host CAMERA 2, providing a perspective from the host's point of view, and Key 0 to select VR controller CAMERA 3, providing a perspective from the VR controller's point of view. Camera selection may also occur using a side camera change button on the controller 20.

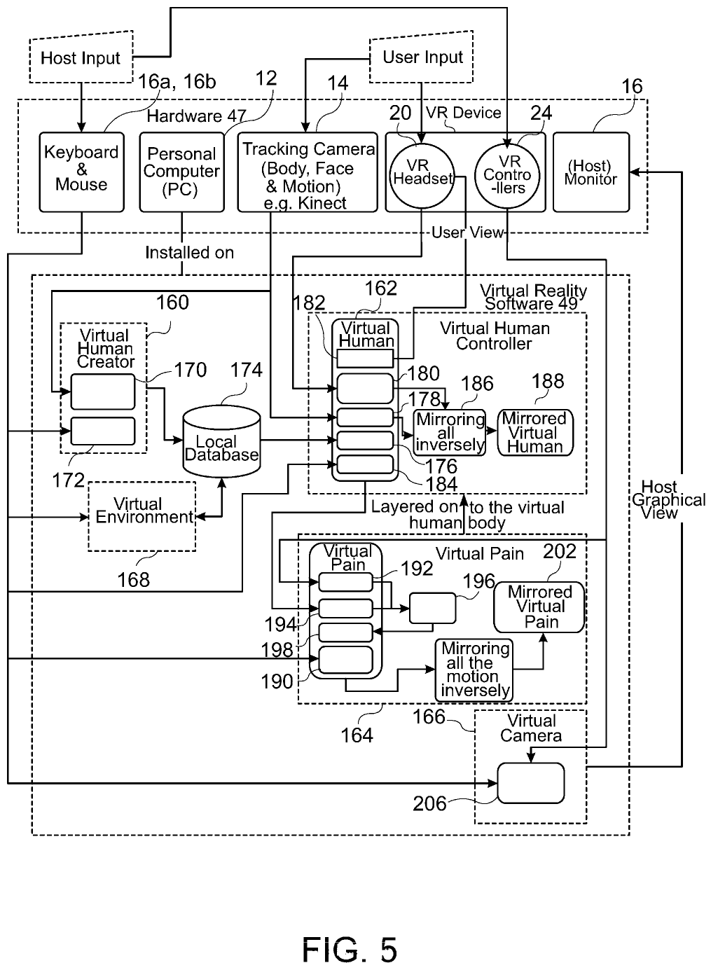

Referring now to FIG. 5, a schematic block diagram of an embodiment of the interoperable hardware and software components of the VR-based system is shown in which previously described hardware components are indicated with the same numerals. The VR software 49 installed on the computer processing system or PC 12 includes various software modules, including a virtual human/user creator module 160, a virtual human/user controller module 162, a virtual pain/condition module 164, a virtual camera module 166 and a virtual environment module 168.

The virtual human/user creator module 160 receives inputs from the tracking camera 14 and renders at sub-module 170 the real images of the user captured by the tracking camera to generate a virtual human/user of the type illustrated, with identifiable user characteristics. These may include body shape, face shape, skin colour, hair style, hair colour, eye colour and any other desired personal user characteristics. In addition, user characteristics of height, weight, body type and gender may be entered by the host via the input hardware 16 in consultation with the user at sub-module 172, with sub-modules 170 and 172 together constituting a rendering engine for rendering the static characteristics of the user. These are then stored in a dedicated user file in secure database 174, which may be a local or remote database.

The virtual human/user controller module 162 generates the virtual user and its mirror image or duplicate for dynamic display through the VR headset, as well as viewing by the host. This is achieved by receiving at sub-module 176 static user data from the database 174, including body and face shape, as well as other user characteristics which have been stored in the user file in the database. A body motion sub-module or sub-class 178 retrieves body motion variables from the tracking camera 14. More specific body position and motion attributes, including head position, head rotation, body position and finger movement data, are retrieved as variables at sub-module 180 from the VR headset 20 and one or more of its associated trackers 26a and 26b and 28a-d.

A dynamic virtual image of the user is generated by combining the above variables to effectively create a virtual user camera at sub-module 182 for display through the VR headset 20. Dynamic feedback from the headset 20 and tracking camera 14 has the effect of dynamically updating the virtual image as seen by the user with minimal latency. The virtual user VR camera position and rotation changes in concert with user induced movement of the VR headset to vary the view of the VR environment. A layering module 184 is operated by the host via inputs 16a, 16b as previously described to enhance the visualisation of the body layer or part requiring treatment, such as skin, nerves, muscles, organs and/or bones.

In conventional virtual reality systems, a mirror within a virtual environment is generated as a plane displaying a reflection of the whole environment. This works like a video projection of the whole environment onto a 2D object within the environment. This creates double the graphical processing requirements, as the engine is trying to render two images of the same environment to show a mirror effect to be displayed on the screen. In the case of a VR headset with two screens, one for each eye, this again doubles the graphical processing requirements, with four environments required to be rendered.

In the present disclosure, a mirroring or inverting sub-module or engine 186 generates an inverse image of the virtual body instead of using a virtual mirror plane, with the same effect of generating a mirrored virtual human or user at 188. This provides flexibility to manipulate the duplicate inverse body, which can be controlled separately from the user body. For example, with a virtual mirror plane it is not possible to see one's back when looking forward. With the duplicate virtual body technique the virtual body can be rotated to allow the user to observe and have explained to them treatments on their back side.

There is further a reduction in graphical processing requirements needed to render the experience, with the graphical processor only needing to render the environment once to be displayed on the screen, or twice in the case of a VR headset. This enhances the performance of the system, reducing lag or latency to ensure that the user's movements are synchronised with their virtual direct and “reflected” representations and increasing the graphic quality of the VR environment.

The virtual pain module 164 is used to generate virtual pain images of the type previously described with input from the host in consultation with the user. The various pain parameters, including pain type, speed and intensity/magnitude, are input and rendered at sub-module 190, with the start and end positions of the pain being entered at 192 and 194 via the VR controllers 24 in the process of finding the right path at 196 and rendering a pain pathway at 198.

A pain mirroring or inverting module 200 generates a mirrored/inverted virtual pain image at 202. The virtual pain images, both direct and inverted, are layered onto the virtual human/user body image generated at the virtual human controller module at 204 and made available to the user as a composite image through the VR headset.

The virtual environment module 168 includes a selection of virtual environments which are selected by the host in consultation with the user, with one or more selected environments being stored in a user file in the database 174. The selected environment is then overlayed/underlayed at the virtual human controller module for display through the VR headset 20 and monitor 16.

The virtual camera module 166 includes a host camera sub-module 206 including the three previously described virtual software cameras 1, 2 and 3 providing the host with views from the host, user and controller perspectives. The sub-module 206 may be controlled by the host via keypad and mouse inputs 16a and 16b as well as via the controller 24 as previously described. The host is able to select camera type, position and rotation variables which will determine the host graphical view on the host GUI 100 on monitor 16.

It will be appreciated that the disclosed treatment system and method may be implemented with other real-and-virtual combined environments and human-machine interactions generated by computer technology and wearables (i.e. XR technologies), such as augmented reality (AR), mixed reality (MR) or any combination of VR, AR and MR.

For example, the term “VR” or “virtual reality” used above can be replaced with the term “XR” or “extended reality” representing the disclosed treatment system and method to be implemented with any of the XR technologies. In particular, a motion tracking device of the XR-based system may be one or more of a Microsoft Kinect 2.0 camera, a Microsoft Azure Kinect camera, a webcam, a mobile phone with LiDAR system or other commercially available tracking cameras, wearables or other sensors to track the user's body and movement. The VR headset 20 may be extended to other XR devices including smartphones, screen and/or projector to display the dynamic virtual image of the user and/or to provide dynamic feedback for dynamically updating the virtual image as seen by the user. It will also be understood that the virtual environment may be created in combination with the real environment to form an XR-type environment such as an AR environment.

In some embodiments, a machine learning software module 51 may be implemented with the XR-based system to facilitate automation of treatment. The machine learning software module 51 may also facilitate generation of treatment reports with analytical data for the treatments that have been done for a user.

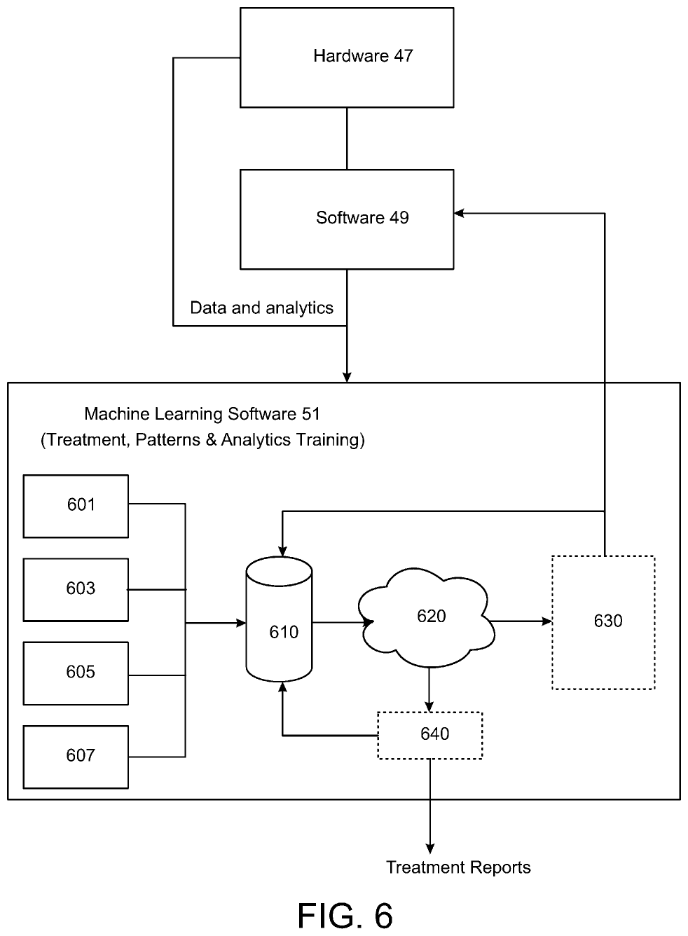

As illustrated in FIG. 6, cloud or local database 610 may collect historical data including, for example, XR hardware data 601 generated from the XR hardware 47, XR software data 603 generated from the XR software 49, user data 605 and/or host data 607 including user's condition history and treatment history (not necessarily the historical data from the current user in treatment). The historical data may be used as ground truth to train a machine learning processor 620 by using, for example, supervised learning techniques (e.g. multilayer perceptron, support vector machine) and/or transfer learning techniques (e.g. contrastive learning approach, or graph matching).

The trained machine learning processor 620 may then be able to provide one or more executable treatment actions 630 for a user currently in treatment based on the input data from the XR hardware 47, the XR software 48, host input and/or user input (e.g. one or more inputs representing one or more attributes of the at least one condition for treatment) from the user currently in treatment. The generated executable treatment actions 630 may then be provided to the XR software 49 for visualisation and/or selectable use by the host and/or the user in treatment. The generated executable treatment actions may also be fed back to the cloud/local database 610 to enrich the historical data for training the machine learning processor 620.

To ensure safety and correctness of the treatment actions, the host may be employed as “human-in-the-loop” to verify and modify the machine generated treatment actions. The verified and/or modified treatment actions may also be fed back to the cloud/local database 610 to enrich the historical data.

The trained machine learning processor 620 may also output analytical data 640 to evaluate one or more treatment results. The analytical data 640 may be used to generate treatment reports which can be provided to the user and/or host. The analytical data 640 may also be fed back to the cloud/local database 610 to enrich the historical data.

### Initial Test Results

Initial development work has established the capability of the virtual reality based treatment system and method generating a seamless virtual reality environment for the user to be immersed in, allowing the user to identify with their self representation as well as with their mirror representation. A pilot sample of four users were tested, all of whom were suffering from chronic pain with a range of diagnoses. All of the users showed immediate transient pain reduction on a single treatment. Of the sample of four, one user subsequently dropped out. The remaining three users responded as follows to treatment over a period of this 10 weeks, with one session per week lasting an average of X minutes:

User 2 received total pain reduction and experienced periods of being completely pain free for the first time in seven years.

User 3 showed reduction in pain severity, reduction in pain locations or extent and reduction on in the impact of pain on their daily living. However, there was still some residual pain, though at reduced levels. It was also noted the residual pain at reduced levels was only located at the site of the injury without any radiating pain.

User 4 had variable results with some improvements. They suffer from hyper-mobile joints and have ongoing pain as they continue to dislocate joints, causing acute proprioceptive pain on an ongoing basis.

Based on these initial results, the applicant is conducting ongoing trials including with regard to intensity and frequency.

It will be appreciated that applications of the treatment method and system are not confined to the treatment of pain, but may potentially be used in treating any condition which can be visualised and depicted. Other applications using neuroplasticity may include rehabilitation therapy in the case of paralysis or palsy, as in the case with stroke sufferers, mental disorders, as well as relaxation therapy using an immersive environment. The condition may relate to amputees, and the treatment may include mental and physical training of amputees, including emulating their lost limb to train their nerves and muscles before using artificial limbs.

It is believed that the onboarding process, including tracking of the entire body of the user and the direct and reflected virtual representations of the user's body, contributes to the user believing, feeling and reacting to the virtual representations/embodiments or avatar as being the real self. It is believed that this serves to engage the brain neuroplasticly to enhance the treatment process.

1. A virtual reality-based treatment system for performing treatment on at least one condition of a subject, comprising:
a virtual reality device arranged to be fitted to the subject and for immersing the subject in a virtual reality environment;
at least one tracking camera configured to capture physical traits and movement of the body of the subject;
a processor communicating with the virtual reality device and the at least one tracking camera;
a monitor in communication with the processor, and including a user interface, wherein
the processor is programmed to:
generate a dynamic virtual representation of the body of the subject based on the captured physical traits and movement of the body of the subject, and to render the virtual representation of the body in the virtual reality environment via the virtual reality device, wherein the dynamic virtual representation is synchronised with the movement of the body of the subject;
generate a virtual representation of the at least one condition of the subject in response to one or more inputs;
overlay or render the virtual representation of the condition of the subject on the virtual representation of the body of the subject; and
receive and process one or more inputs representing one or more attributes of the condition to adjust the virtual representation of the condition of the subject in the virtual reality environment to thereby assist the subject to visualise and resolve the condition.

2. A method of performing a treatment on at least one condition of a subject in an immersive virtual reality environment, comprising:
capturing physical traits and movement of the body of the subject;
generating a dynamic virtual representation of the body of the subject based on the captured physical traits and movement of the body of the subject;
rendering the dynamic virtual representation of the body of the subject in the virtual reality environment, wherein the dynamic virtual representation is synchronised with the movement of the body of the subject;
generating a virtual representation of the at least one condition of the subject in response to one or more inputs;
overlaying or rendering the virtual representation of the condition of the subject on the virtual representation of the body of the subject; and
receiving and processing one or more inputs representing one or more attributes of the condition to adjust the virtual representation of the condition of the subject in the virtual reality environment to thereby assist the subject to visualise and resolve the condition.

3. The method of claim 2, further comprising:
generating virtual representations of multiple layers or components of the virtual body selected from at least two of a skin layer or component, a muscle layer or component, a nerves layer or component, an organs layer or component, a vascular layer or component, a respiratory layer or component and a skeleton layer or component; and
enabling switching between virtual representations of the layers or components.

4. The method of claim 2, wherein the visual representations of the attributes of the condition include at least two of location, start point, end point, depth, intensity, size, speed, direction, frequency, temperature as indicated by colour, and type as indicated by symbols.

5. The method of claim 2, wherein the captured physical traits include at least three of body shape, face shape, skin colour, hair colour/style, eye colour, height, weight, and gender.

6. The method of claim 2, wherein the step of generating virtual representations of the body of the subject includes generating selectable or interchangeable direct self and mirror self-representations of the subject.

7. The method of claim 6, wherein the mirror representations of the subject are generated by generating an inverse image of the subject as opposed to using a virtual mirror plane.

8. The method of claim 6, wherein the step of generating virtual representations of the at least one condition of the subject includes generating direct and mirror representations of the at least one condition and overlaying the direct and mirror representations of the condition on the respective direct and mirror representations of the subject.

9. The method of claim 8, wherein the mirror representations of the at least one condition are generated by generating an inverse image of the at least one condition as opposed to using a virtual mirror plane.

10. The method of claim 2, wherein immersing the subject in the virtual reality environment comprises enabling selection of an immersive environment for occupation by the virtual representation of the subject from a plurality of different immersive environments, the plurality of different immersive environments including onboarding (neutral), underwater, green field, snow, mountain, forest, tropical island, and desert.

11. The method of claim 2, further comprising generating a virtual representation of the body of a host or treatment provider, based on the captured physical traits and movement of the body of the host, and rendering the virtual representation of the body of the host in the virtual reality environment.

12. The method of claim 2, wherein the condition is one of pain, chronic pain, a physical or mental ailment or disability including amputeeism and various levels of paralysis or palsy, and a physical or mental state which requires enhancing or therapy including muscle condition, mental acuity, or stress.

13-16. (canceled)

17. A virtual reality-based treatment system for performing treatment on at least one condition of a subject, comprising:
a virtual reality device arranged to be fitted to the subject and for immersing the subject in a virtual reality environment;
at least one tracking camera configured to capture physical traits and movement of the body of the subject;
a processor communicating with the virtual reality device and the at least one tracking camera;
a monitor in communication with the processor, and including a user interface, wherein
the processor is programmed with a plurality of software modules to generate a dynamic virtual representation of the body of the subject based on the captured physical traits and movement of the body of the subject, and to render the virtual representation of the body in the virtual reality environment via the virtual reality device, wherein the dynamic virtual representation is synchronised with the movement of the body of the subject, the software modules including;
a virtual subject creator module to capture physical traits of the body of the subject and render a virtual subject including those traits;
a virtual subject controller module to capture movement of the body of the subject and render a moving virtual subject using the virtual subject from the virtual human creator module;
a virtual condition module to generate a virtual representation of the at least one condition of the subject in response to one or more inputs, and layer it on the moving virtual subject; and
a virtual environment module for providing a selectable virtual environment for the subject.

18. An extended reality (XR) based treatment system for performing treatment on at least one condition of a subject, comprising:
an XR device arranged to be fitted to the subject and for engaging the subject in an XR environment;
at least one motion tracking device configured to capture physical traits and movement of the body of the subject;
a processor communicating with the XR device and the at least one motion tracking device;
a monitor in communication with the processor, and including a user interface, wherein
the processor is programmed to:
generate a dynamic virtual representation of the body of the subject based on the captured physical traits and movement of the body of the subject, and to render the virtual representation of the body in the XR environment via the XR device, wherein the dynamic virtual representation is synchronised with the movement of the body of the subject;
generate a virtual representation of the at least one condition of the subject in response to one or more inputs;
overlay or render the virtual representation of the condition of the subject on the virtual representation of the body of the subject; and
receive and process one or more inputs representing one or more attributes of the condition to adjust the virtual representation of the condition of the subject in the XR environment to thereby assist the subject to visualise and resolve the condition.

19. (canceled)

20. An extended reality (XR) based treatment system for performing treatment on at least one condition of a subject, comprising
an XR device arranged to be fitted to the subject and for engaging the subject in an XR environment;
at least one motion tracking device configured to capture physical traits and movement of the body of the subject;
a processor communicating with the XR device and the at least one motion tracking device;
a monitor in communication with the processor, and including a user interface, wherein
the processor is programmed with a plurality of software modules to generate a dynamic virtual representation of the body of the subject based on the captured physical traits and movement of the body of the subject, and to render the virtual representation of the body in the XR environment via the XR device, wherein the dynamic virtual representation is synchronised with the movement of the body of the subject, the software modules including;
a virtual subject creator module to capture physical traits of the body of the subject and render a virtual subject including those traits;
a virtual subject controller module to capture movement of the body of the subject and render a moving virtual subject using the virtual subject from the virtual human creator module;
a virtual condition module to generate a virtual representation of the at least one condition of the subject in response to one or more inputs, and layer it on the moving virtual subject; and
an XR environment module for providing a selectable XR environment for the subject.

21. A method of performing a treatment on at least one condition of a subject in an extended reality (XR) environment comprising:
capturing physical traits and movement of the body of the subject;
generating a dynamic virtual representation of the body of the subject based on the captured physical traits and movement of the body of the subject;
rendering the dynamic virtual representation of the body of the subject in the XR environment, wherein the dynamic virtual representation is synchronised with the movement of the body of the subject;
generating a virtual representation of the at least one condition of the subject in response to one or more inputs;
overlaying or rendering the virtual representation of the condition of the subject on the virtual representation of the body of the subject; and
receiving and processing one or more inputs representing one or more attributes of the condition to adjust the virtual representation of the condition of the subject in the XR environment to thereby assist the subject to visualise and resolve the condition.

22. (canceled)

23. The extended reality (XR) based treatment system of claim 18, wherein the extended reality based treatment system is selected from the group comprising at least one of virtual reality (VR), augmented reality (AR), and mixed reality (MR).

24. The XR-based treatment system according to claim 18, further comprising:
a database for collecting historical data; and
a machine learning processor;
wherein the historical data is used to train the machine learning processor so that the machine learning processor generates one or more executable treatment actions based on the one or more inputs representing one or more attributes of the at least one condition of the subject; and
wherein the generated one or more executable treatment actions are provided to the processor for visualisation and resolving the condition.

25. The XR-based treatment system according to claim 24, wherein the historical data includes one or more of XR hardware data, XR software data, user data and host data.

26. The XR-based treatment system according to claim 24, wherein the generated one or more executable treatment actions are fed back to the database.

27. The XR-based treatment system according to claim 24, wherein the trained machine learning processor further generates analytical data to evaluate one or more treatment results, and wherein the generated analytical data is fed back to the database.
